(19) United States
(12) Patent Application Publication
(10) Pub. No.: US 2024/0161107 A1
(43) Pub. Date: May 16, 2024

(54) NFT TRANSACTION METHOD AND TRANSACTION SYSTEM RELATING TO BIOLOGICAL FEATURE

(71) Applicants: TSUNG-CHI CHEN, New Taipei City (TW); YING-CHEN YANG, New Taipei City (TW)

(72) Inventor: TSUNG-CHI CHEN, New Taipei City (TW)

(21) Appl. No.: 18/388,340

(22) Filed: Nov. 9, 2023

(30) Foreign Application Priority Data
Nov. 11, 2022 (TW) 111143294

(51) Int. Cl. G06Q 20/38; G06Q 20/40

(52) U.S. Cl. CPC G06Q 20/389; G06Q 20/401

(57) ABSTRACT

NFT transaction method and system relating to biological feature include: receiving, at a biological asset module of an NFT transaction system, a biological cell or tissue from a provider; obtaining, at the biological asset module, the biological feature from the biological cell or tissue; producing, at a digital asset module of the NFT transaction system, a digital creation based on the biological feature; minting, at the digital asset module, a to-be-traded NFT corresponding to the digital creation; evaluating and updating, at a transaction module of the NFT transaction system, a market price of the to-be-traded NFT based on a market condition of the biological feature; and conducting a first transaction matchmaking for the to-be-traded NFT on a virtual transaction platform based on the market price.

### CROSS REFERENCE TO PRIORITY APPLICATIONS

The present application claims the priority of TW application No. 111143294 by CHEN et al., titled “NFT TRANSACTION METHOD AND TRANSACTION SYSTEM CORRESPONDING TO BIOLOGICAL FEATURE”, filed on Nov. 11, 2022 at the Taiwan Intellectual Property Office, TIPO. The disclosure of which is incorporated herein in its entirety.

### FIELD OF THE INVENTION

The invention relates to a data processing method and system thereof, and more particularly, to an NFT transaction method and transaction system relating to biological feature.

### BACKGROUND OF THE INVENTION

Current blockchain technology is based on a decentralized peer-to-peer network and combines cryptography principles, time series data, and consensus mechanisms. The transaction information is stored in decentralized nodes that are dispersed in different locations. The data series in these decentralized nodes are consistent and coherent, and the information can be verified, traced, and is difficult to tamper with. NFTs (Non-Fungible Tokens) are based on blockchain technology, and an NFT is indivisible, irreplaceable, and unique. Every NFT represents a unique, one and only digital asset, which can be a photo, a piece of text, a painting, a piece of music, etc. It can be tracked and traded over blockchain. Therefore, the authenticity of an NFT can be proved.

However, NFT transactions can be manipulated by humans and the price can be hyped easily. As such, it is difficult to evaluate or estimate the true market price of the digital asset. Even if transaction terms and profit allocation ratios are defined in the smart contract, still, only virtual transactions can be regulated by the smart contract terms.

The issue of profit allocation is more prominent in the pharmaceutical business. The research and development of drugs or medical materials require a significant invest in time and money. The distribution of capital and profit are not always fair and reasonable. The pharmaceutical companies usually receive most of the profits, while the test subjects and donors are usually not reasonably compensated. This is one of the reasons why people are not willing to provide or deposit biological samples. Further, for biological samples processed through traditional fashion, their liquidity on the commercial market is quite limited. Pharmaceutical companies or research institutes often cannot obtain the samples they need in time. The business relating to trading biological samples grows slowly, which results in a low successful rate of drug development.

### SUMMARY OF THE INVENTION

The purpose of the invention is to solve the problems in the prior art. The NFT transaction method and transaction system relating to biological feature perform the process of minting a to-be-traded NFT after a digital creation is obtained or received. The NFT undergoes a transaction matchmaking on a virtual transaction platform based on a market price corresponding to a market condition.

According to one aspect of the invention, an NFT transaction method relating to a biological feature is provided. The method includes: (1) receiving, at a biological asset module of an NFT transaction system, a biological cell or tissue from a provider; (2) obtaining, at the biological asset module, the biological feature from the biological cell or tissue; (3) producing, at a digital asset module of the NFT transaction system, a digital creation based on the biological feature; (4) minting, at the digital asset module, a to-be-traded NFT corresponding to the digital creation; (5) evaluating and updating, at a transaction module of the NFT transaction system, a market price of the to-be-traded NFT based on a market condition of the biological feature; and (6) conducting a first transaction matchmaking for the to-be-traded NFT on a virtual transaction platform based on the market price.

In one embodiment, the NFT transaction method further includes: (7) conducting a second transaction matchmaking for the biological cell or tissue on a physical transaction platform based on a fiat currency price reflecting the market price.

In one embodiment, the NFT transaction method further includes: (8) creating a smart contract for converting the market price into the fiat currency price based on an exchange rate of the virtual transaction platform.

In one embodiment, the NFT transaction method further includes: (9) creating a smart contract for sharing a profit portion to the provider according to a sharing ratio when the first matchmaking is successful.

In one embodiment, in step (9), the smart contract is created to include a monetary flow information that directs to a financial account corresponding to the provider.

In one embodiment, the NFT transaction method further includes: (10) generating a digital form corresponding to the provider, and the digital form includes at least one public information that is de-identified, and the biological feature and the digital creation are linked to the digital form.

In one embodiment, in step (1), the biological cell or tissue includes but not limited to blood, cord blood, immune cells, stem cells, skin cells, chondrocytes, cartilage, or cancer tissues.

In one embodiment, step (3) further includes: (31) setting a numerical range corresponding to the biological feature; (32) forming a plurality of creation units, wherein each creation unit comprises one or more numerical values within the numerical range; and (33) assembling the creation units to form the digital creation.

In one embodiment, in step (3), the digital creation includes a music melody, a digital image, or a digital video.

In one embodiment, in step (5), the market condition includes a search number of the biological feature on the virtual transaction platform, a number of the to-be-traded NFT on the virtual transaction platform, a historical transaction number of the to-be-traded NFT on the virtual transaction platform, or a number of the biological feature corresponding to the provider.

In one embodiment, in step (4), the biological feature is used as an authentication information for retrieving a blockchain private key of the to-be-traded NFT.

In one embodiment, the NFT transaction method further includes: encrypting the authentication information.

According to another aspect of the invention, an NFT transaction method relating to a biological feature is provided. The method includes: (1) receiving a digital creation which is produced based on the biological feature of a biological cell or tissue; (2) minting a to-be-traded NFT corresponding to the digital creation; (3) evaluating and updating a market price of the to-be-traded NFT based on a market condition of the biological feature; and (4) conducting a first transaction matchmaking for the to-be-traded NFT on a virtual transaction platform based on the market price.

In one embodiment, the NFT transaction method further includes: (5) conducting a second transaction matchmaking for the biological cell or tissue on a physical transaction platform based on a fiat currency price reflecting the market price.

In one embodiment, in step (2), the biological feature is used as an authentication information to retrieve a blockchain private key of the to-be-traded NFT.

In one embodiment, the NFT transaction method further includes: encrypting the authentication information.

According to yet another aspect of the invention, an NFT transaction system relating to a biological feature is provided. The system includes: a digital asset module and a transaction module. The digital asset module is used for obtaining a digital creation which is produced based on the biological feature of a biological cell or tissue and for minting a to-be-traded NFT corresponding to the digital creation. The transaction module is used for listing the to-be-traded NFT, evaluating and updating a market price of the to-be-traded NFT based on a market condition of the biological feature, and conducting a first transaction matchmaking for the to-be-traded NFT based on the market price.

In one embodiment, the transaction module is further used for conducting a second transaction for the biological cell or tissue based on a fiat currency price reflecting the market price.

In one embodiment, the digital asset module is further used for configuring the biological feature as an authentication information for retrieving a blockchain private key of the to-be-traded NFT.

In one embodiment, the digital asset module is further used for encrypting the authentication information.

The NFT transaction method and transaction system relating to biological feature performs the process of minting the to-be-traded NFT after obtained or received the digital creation. The value of the NFT is evaluated based on the market condition of the biological feature and the NFT is traded on the virtual transaction platform, which includes the advantages at least including protecting the privacy of the provider, having the NFT's value matching with the market condition, making the transaction secure and transparent, and facilitating the commercialization of the biological features.

### BRIEF DESCRIPTION OF THE DRAWINGS

Many aspects of the embodiments can be better understood with reference to the following drawings. The components in the drawings are not necessarily drawn to scale, the emphasis instead being placed upon clearly illustrating the principles of the embodiments. Moreover, in the drawings, like reference numerals designate corresponding parts throughout the several views.

FIG. 1 is a flow chart of an NFT transaction method according to one embodiment of the invention;

FIG. 2 is a block diagram of an NFT transaction system according to one embodiment of the invention;

FIG. 3 is a flow chart of the producing method of the digital creation according to one embodiment of the invention;

FIG. 4 is a flow chart of an NFT transaction method according to another embodiment of the invention;

FIG. 5 is a flow chart of an NFT transaction method according to yet another embodiment of the invention;

FIG. 6 is a flow chart of an NFT transaction method according to a further embodiment of the invention; and

FIG. 7 is a block diagram of an NFT transaction system according to a further embodiment of the invention.

### DETAILED DESCRIPTION OF THE INVENTION

In the NFT transaction method and transaction system relating to biological feature according to the embodiments of the invention, a particular digital creation that is unique to the provider is produced. The value of the NFT is evaluated according to the market condition of the biological feature, and then can be traded accordingly. The merits of the embodiments of the invention can be shown at least in that the privacy of the provider is protected, the value of the NFT matches with the market's actual condition, and the transaction process is transparent, which facilitates the commercialization of the biological features. The NFT transaction method and transaction system of the embodiments of the invention will be elaborated in below with accompanying drawings to show the technical features of the present invention, and a person skilled in the art can better understand the content of the invention.

It should be noted that the embodiments detailed in the below are for elaboration purpose only, not for limiting the invention. Unless particularly specified or clearly defined, the orders of the method steps are not limited to those disclosed in the embodiments. Further, a person skilled in the art could make alterations or modifications based on the disclosure of the embodiments of the invention, and these changes will still be regarded as part of the invention.

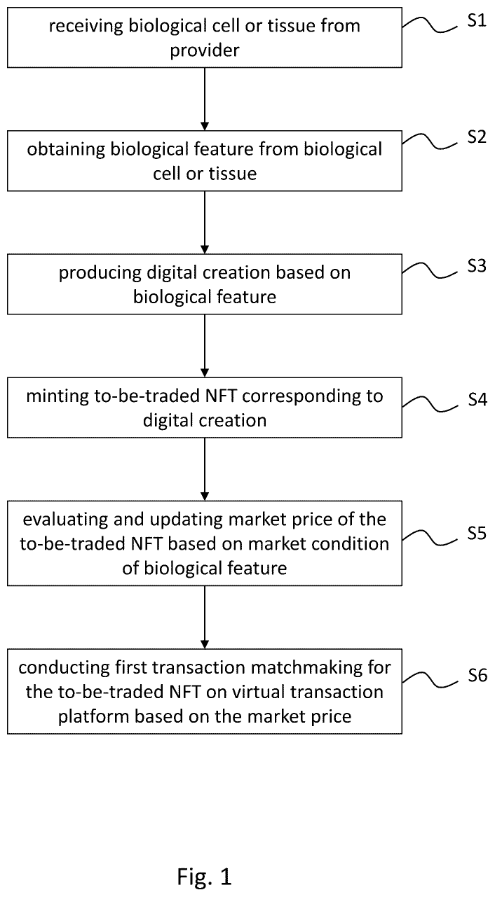

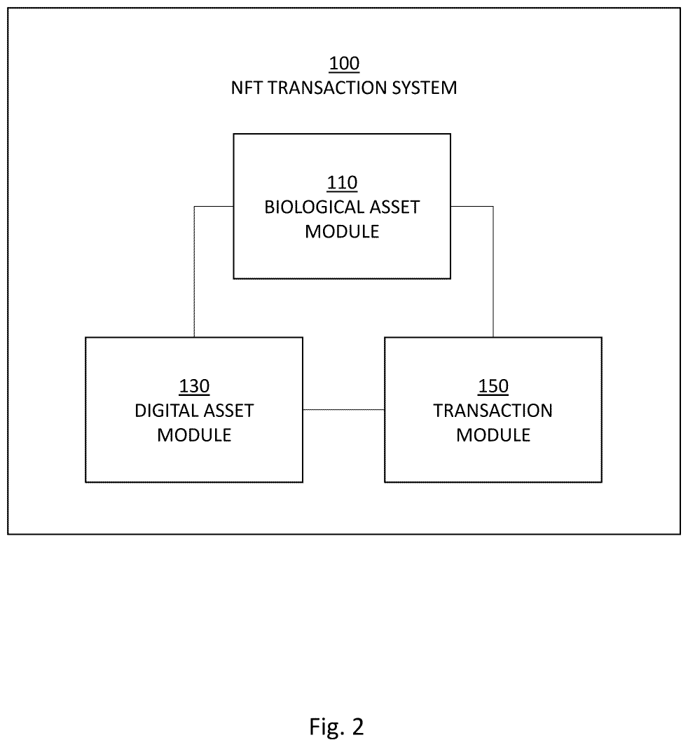

Please refer to FIG. 1 and FIG. 2 at the same time. FIG. 1 is a flow chart of an NFT transaction method according to one embodiment of the invention. FIG. 2 is a block diagram of an NFT transaction system according to one embodiment of the invention. The NFT transaction method of the embodiment is implemented by the NFT transaction system 100 as an example.

The NFT transaction system 100 includes a biological asset module 110, a digital asset module 130, and a transaction module 150. The biological asset module 110 is used for receiving a biological cell or tissue from a provider and for obtaining a biological feature from the biological cell or tissue. The digital asset module 130 is used for receiving the biological feature and producing a digital creation based on the biological feature, and further for minting a to-be-traded NFT (will be mentioned as “the NFT” in below description) based on the digital creation. The transaction module 150 is used for listing the NFT and for evaluating and updating a market price of the NFT. The transaction module 150 is also used for conducting a first transaction matchmaking for the NFT based on the market price.

The NFT transaction system 100 can be exemplified by but not limited to computer(s), server(s), data center(s), cloud virtual machine(s), or mainframe(s). The NFT transaction system 100 at least includes a storage device, a memory, a data bus, an output device, an input device, a central processing unit, and a graphic processing unit. A person who is skilled in the field of computer science or engineering would understand the structural composition or architecture of these components, and their detail is not limited in the present invention. The central processing unit executes the operating system to operate the NFT transaction system 100. The storage device can be a traditional floppy disk drive, an optical disk drive, a hard disk drive, a solid-state storage device, or other storage means used for storing the operating system and software, programs, database, and other software packages necessary to the modules 110, 130, and 150 of the NFT transaction system 100. The memory is, for example, a dynamic random access memory, which can also be a multi-stage memory, such as a hierarchy of dynamic random access memory and static random access memory. The memory is used for storing commands and data to facilitate the operations or tasks of the system 100.

Each module 110, 130, or 150 of the NFT transaction system 100 can be set up in the same geographic location, or, in a different embodiment, they can be in different locations. When the modules 110, 130 and 150 are in different locations, they can communicate with each other through the Internet, virtual private network (VPN), 5G, LTE, or other applicable network architectures. The modules in the system 100, namely the biological asset module 110, the digital asset module 130, and the transaction module 150, may each include but not limited to one or more computers or servers. The computers or servers of the same module 110, 130, or 150 can be located in the same datacenter at a designated geographic location. In another embodiment, the computers or servers of the same module 110, 130, or 150 can also be located in different datacenters. Further, they can also be set up on one or more virtual machines running on the same or different cloud platforms. To elaborate in detail, in one embodiment, the biological asset module 110 is in the server room of institution A, the digital asset module 130 is in the server room of institution B, and the transaction module 150 is in the server room of institution C. In another embodiment, two of the three modules 110, 130, and 150 are in the same institution. For example, the biological asset module 110 and the digital asset module 130 are in the same institution, or the digital asset module 130 and the transaction module 150 are in the same institution, or the biological asset module 110 and the transaction module 150 are in the same institution. The geographic locations of the modules 110, 130, and 150 and their computer types (e.g. a server, a virtual machine, or other types) can be configured and adjusted according to actual needs. These details are not limited by the present invention.

Each module 110, 130, or 150 can further include an interface for interacting with users. The interface can be a web page presented by a web browser or a user interface of a program running on the operating system. A user can operate the modules collectively or individually and locally or remotely through personal computer, laptop, smartphone, tablet, etc.

The NFT transaction method of the present embodiment includes step S1 to step S6, as shown in FIG. 1.

In step S1, the biological cell or tissue from the provider is received. In this step, the biological cell or tissue include but not limited to blood, cord blood, immune cells, stem cells, skin cells, chondrocytes, cartilage, or cancer tissues. Any other cells or tissues that are biomedically valuable can be used herein as the biological cell or tissue. In the present embodiment, the biological asset module 110 can be a biobank or a biological depository institution, and it receives the biological cell or tissue. The provider can create a virtual account at the biological asset module 110. In the virtual account, all the information stored therein is de-identified information, so the identity of the provider cannot be learnt from the information. The de-identified information can be exemplified by attributes like age, blood type, gender, disease, race, nationality, height, and weight etc. Additionally, the virtual account stores a link code directing to a financial account corresponding to the provider. Even when a financial operation is conducted, only the content of the transaction can be acquired from the virtual account, such as the code of the NFT, the code of the traded biological cell or tissue, transaction amount, payee's code, etc. The link code is used for notifying the financial institution where the provider's financial account is located to perform cash flow operations such as receiving remittances. The financial transaction related information of the provider, such as information for perform remittances or withdrawals of legal tenders, is stored in the provider's financial account.

Next, in step S2, the biological feature of the biological cell or tissue is obtained. In the present embodiment, the biological feature is obtained at the biological asset module 110. The biological feature usable herein includes but not limited to DNA sequences of cells or other biologically relevant traits or unique characteristics. The biological feature is used to obtain or identify individual identity, specific diseases, drug responses, or other biomedically significant information. The biological asset module 110 performs DNA extraction and sequencing. The content of these techniques is not limited to the present invention. The biological asset module 110 then provides the obtained biological feature to the digital asset module 130. Optionally, the biological asset module 110 can also provide a custom code generated by the provider to the digital asset module 130. In one embodiment, the biological asset module 110 further includes, for example, a freezer that receives and stores the biological cell or tissue, a robotic arm, various analysis instruments, various detection instruments, and other related or similar devices. The applicable devices and their configurations are not limited to the embodiments of the invention.

Further in step S3, a digital creation is produced. The digital creation is produced based on the biological feature through a producing method. The producing method is elaborated in below detail.

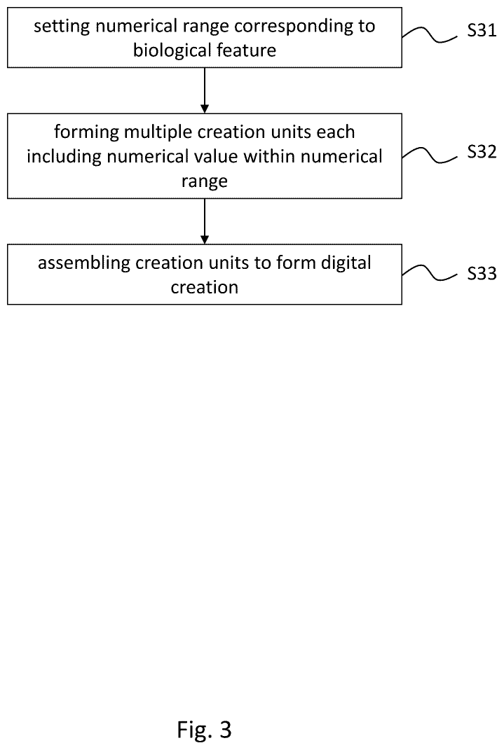

Please refer to FIG. 3, which is a flow chart of the producing method of the digital creation according to one embodiment of the invention. First, step S31 is performed, where a numerical range corresponding to the biological feature is set. In one embodiment, the expressions of the biological feature can be converged or organized into an expression range. The maximum value and the minimum value of the expression range can be obtained accordingly. The maximum value is set as the upper limit of the numerical range, and the minimum value is set as the lower limit of the numerical range. In one embodiment, the numerical range represents a brightness range of light, where the lower limit is the darkest brightness and the upper limit is the maximum brightness. In another embodiment, the numerical range represents a wavelength range of light, where the upper limit is set to be a first wavelength and the lower limit is set to be a second wavelength of light. The numerical range denotes the change of colors. In yet another embodiment, the numerical range represents a sound frequency range, where the upper limit is set to be a treble frequency and the lower limit is set to be a bass frequency. The numerical range denotes the change of sound pitches. Then the producing method moves on to step S32, in which numerous creation units are formed. Each creation unit includes one or more numerical values within the numerical range and can be exemplified by a pixel or a waveform of sound. Then in step S33, the creation units are assembled to form the digital creation. In the example that the creation units are pixels, the digital creation is a digital image, and when the creation units are soundwaves, the digital creation is a music melody.

Please refer to FIG. 1 and FIG. 2 again. In the present embodiment, after the digital asset module 130 receives the biological feature, it produces the digital creation based on the biological feature through the producing method. In addition to the digital image and music melody, the digital creation can also be in the form of video clips and other personalized artistic expressions. The digital asset module 130 can also have the digital creation manufactured into physical objects, such as printed images embedded on necklaces or keychains, or music boxes playing the music melody. The digital asset module 130 may include creative software, such as software applied with AI technology or digital creativity database. The digital asset module 130 may further include musical instrument for creating the digital creation and output device for outputting the digital creation. The output device can be exemplified but not limited to speaker, screen, LED light, and other devices that can be used to produce sounds and/or lights. After the digital creation is produced, it can be sent to the following step S4 of minting the NFT. Optionally, the digital creation can be encrypted by one or more encryption mechanisms, and then the encrypted digital creation is provided to step S4.

The NFT transaction method continues to step S4, minting the NFT corresponding to the digital creation. In the present embodiment, the NFT is minted in the digital asset module 130. Here “NFT” is the abbreviation of Non-Fungible Token and is, for example, generated over the blockchain based on the ERC-721 standard. The NFT uniquely corresponds to the digital creation, and its minting process is not limited in the invention. If a minting process generates an NFT that is traceable, difficult to tamper with, can be validated in real time, and can be used for proofing the genuineness of the digital creation, such process is applicable in the present invention.

The digital asset module 130 may further include a blockchain module for minting the NFT. The biological feature is used as an authentication information of a blockchain private key of the NFT, so it can be used for retrieving the blockchain private key. While minting the NFT, the digital asset module 130 uses the biological feature as the authentication information of the blockchain private key. When a security information related to the NFT is lost, the biological feature can be used to salvage the blockchain private key. The security information includes but is not limited to blockchain wallet address, blockchain private key, and backup phrase. Even in the case that the user loses the security information, the digital asset can still be recovered. Since the biological feature is the unique information of the provider, it can be used to verify the identity of the provider. In one embodiment, the biological feature of the provider is a DNA sequence. At least a portion of the sequence can be used as the authentication information to retrieve the blockchain private key. Since a third party is difficult to obtain the provider's biological feature as a means for retrieving the blockchain private key, the privacy can be protected and the security can be enhanced.

Furthermore, since the biological feature can be used as the authentication information, the NFT transaction method further includes the following step: encrypting the authentication information. In one embodiment, the digital asset 130 is further used for encrypting the authentication information.

The process of encrypting the authentication information is a process to transform the plaintext information into ciphertext that is difficult to read. Examples of the data centric encryption method includes: (1) symmetric key encryption, i.e., private key encryption, where keys for encryption and decryption are identical and examples of the symmetric algorithm includes DES, 3DES, AES, and etc.; (2) asymmetric key encryption, i.e., public key encryption, where the paired public and private keys are used for encryption and decryption, data encrypted with the public key can only be decrypted with the paired private key, data encrypted with the private key can only be decrypted with the paired public key, public key can be made public and the private key is kept by individual, and examples includes RSA, DSA, and etc.; (3) hash encryption technology, a.k.a. one-way encryption technology, where data or information of any length is converted to a scrambled code (of 128 bits for example) called hash value, and to be more specific, normally a one-way hash function is used to receive bits of any length and generate a unique fixed-length fingerprint (usually 128 bits) and the process is one-way since the information cannot be restored from the fingerprint, and there will be no two messages having the same hash value, so such mechanism can be used for security, identity authentication, and information integrity check, examples of hash encryption include MD2, MD4, MD5, SHA-1, and etc.

In addition, modern dynamic encryption technology uses the combination of symmetric encryption, asymmetric encryption, and hash encryption. The combination of the encryptions utilizes the advantages of each encryption algorithm and minimizes their drawbacks. Normally the strength of encryption is related to the length of the key and the means of generating and safekeeping the key, yet for further security, the data can be fragmented. The fragmented data can be protected by fragmentation encryption technology, blockchain management technology, multi-node multi-backup technology, and smart contract. By using encryption technologies, the key and/or the password could not be cracked easily, and the transmission process can be protected. Moreover, post-quantum cryptography, PQC algorithm can also be implemented to encrypt the plaintext to further increase security.

In the process of encrypting the authentication information, one or more encryption technologies can be used to format the information corresponding to the biological feature. The formatted information is used as the basis for identity authentication, which is regarded as authentication information. In the present embodiment, the biological feature is used as the means to authenticate the identity and retrieve the private key. The biological feature, such as DNA information, is encrypted to ensure its security and traceability.

Another encryption method is related to fractal technology which can increase the security strength even further. The method includes the following example steps. In one embodiment, after the information corresponding to the biological feature (such as DNA information) is digitized and converted into a file, the file is encrypted. Then a fractal tree is established, and an index is created. The file is then fractured into multiple fragment data. After the file is encrypted and fractured, the root hash value and the hash value of each fragment data are obtained. The root hash value is recorded to the blockchain for decentralization, and the fragmented data and their corresponding hash values are stored in distributed storage nodes. In fractal encryption technology, one encryption algorithm is secured by another. Even if one fragment data is cracked, the whole information cannot be obtained therefrom. After the information is fractured and intensely encrypted, the fragments are transmitted through point-to-point communication, which effectively eliminates the possibility of hackers intercepting and stealing data during transmission. In addition, the multi-node multi-backup technology can prevent information from being damaged or even lost, such as accidentally deleted by human error or lost during natural disasters.

In the present embodiment, the digital asset module 130 is at least used for performing step S3 and step S4. The submodule for step S3 and the submodule for step S4 can be integrated in the same computer system, server, or mainframe and located at the same geographic location, such as the server room of one institution. However, in another embodiment, the two submodules are in different geographic locations and connected by cloud or by local network. In such configuration, the digital creation can be encrypted with one or more encryption mechanisms in one submodule and then transmitted to another submodule through network, which fortifies the security measures.

The NFT transaction method continues to step S5 and step S6. In step S5, a market price of the NFT is evaluated and updated based on a market condition of the biological feature. In step S6, a first transaction matchmaking for the NFT is conducted on a virtual transaction platform based on the market price.

In the present embodiment, after the NFT is minted, it is listed and issued by the transaction module 150. The transaction module 150 includes the virtual transaction platform and is used for evaluating the market price based on the market condition. The market condition includes but is not limited to a search number of the biological feature on the virtual transaction platform, a number of the to-be-traded NFT on the virtual transaction platform, a historical transaction number of the to-be-traded NFT on the virtual transaction platform, or a number of the biological feature corresponding to the provider. When there is a higher search number, a larger number of the to-be-traded NFT, a larger historical transaction number, or a larger number of the biological feature to one provider (means the provider discloses more biological features), the biological feature is more valuable, the biological cell or tissue is more valuable, and the corresponding to-be-traded NFT is also more valuable. Therefore, the NFT has a higher rate of success for matchmaking and has a higher price. Further, the provider can choose to issue the NFT anonymously or non-anonymously on the virtual transaction platform.

After the first transaction matchmaking on the virtual transaction platform is completed, the NFT transaction method relating to the biological feature according to the present embodiment is completed.

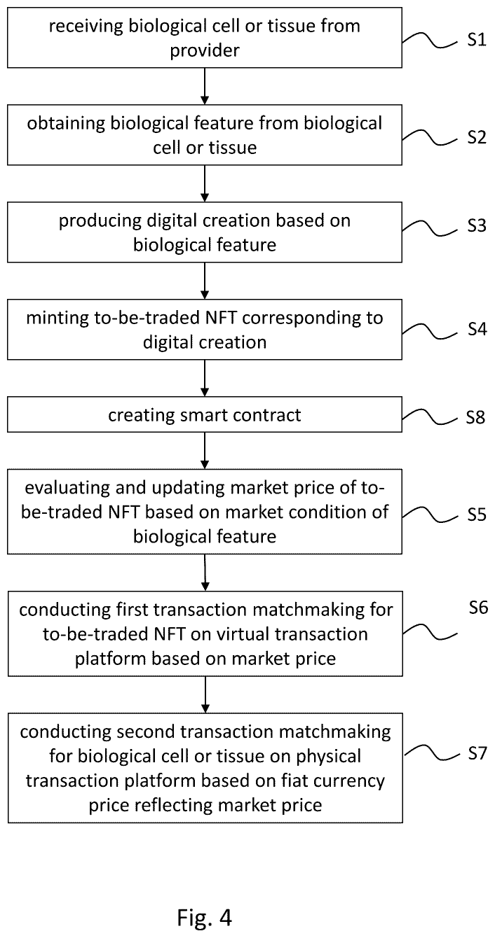

Please refer to FIG. 4, which is a flow chart of an NFT transaction method according to another embodiment of the invention. As an example, the NFT transaction method of the present embodiment is implemented by the NFT transaction system 100 relating to FIG. 2. Step S1 through step S6 are the same as the above-mentioned embodiment relating to FIG. 1 and will not be repeated here.

The NFT transaction method of the present embodiment further includes step S7 and step S8. Step S7 can be executed after step S6 for example. In step S7, a second transaction matchmaking for the biological cell or tissue is conducted on a physical transaction platform based on a fiat currency price reflecting the market price. Step S8 can be performed between step S4 and step S5 as an example. In step S8, a smart contract is created. Based on the content of smart contract, a converting step can be performed prior to step S7. In the converting step, the market price is converted into the fiat currency price based on an exchange rate of the virtual transaction platform. The fiat currency price is then linked to the market price. In addition to the rate exchanging operation, the smart contract can also specify an initial price of NFT, authorization of the NFT, fund transfer operation, profit sharing operation, and other related operations.

Moreover, the smart contract is created to include a monetary flow information that directs to a financial account corresponding to the provider. When the first transaction matchmaking is successful, a profit portion of the payment received by the seller will be shared to the provider according to a sharing ratio defined in the smart contract. That is, the provider will be provided with reasonable profits, and the problem of the providers not being reasonably compensated can be resolved. Additionally, the buyer can only learn the public information of the virtual account that is de-identified and cannot trace back to the identity of the provider. The privacy is therefore been protected and security is improved.

The transaction module 150 includes, in one embodiment, both the virtual transaction platform and the physical transaction platform for conducting virtual end transaction and physical end transaction respectively. Regarding the virtual end transaction, the buyers are for example NFT collectors who choose to purchase the NFT for the particularity of the biological cell or tissue or the rarity of the NFT itself. They may merely add the NFT to their collection or wait for a higher price to sell it. As a result, the NFT can have good market liquidity. The virtual end transaction also allows the buyers to make payments in virtual currency on the virtual transaction platform, which increases the convenience for the buyers.

As for the physical end transaction, the buyers are for example research institutions. They can search and pick the appropriate biological cell or tissue on the physical transaction platform and send the information of the NFT corresponding to the biological cell or tissue to the transaction module 150 or the digital asset module 130. The module 150 or 130 is then ready for physical transaction. In one embodiment, if the second transaction matchmaking is successful, the buyers (research institutions) will notify the transaction module 150, and the module 150 subsequently provides the content of the transaction (for example, the code of the traded biological cell or tissue, the fiat currency price, the payer's code, the payee's code etc.) to the financial account of the buyer. The module 150 notifies the buyer's financial account to send the remittance and notifies the provider's financial account to be ready to receive the payment. After the remittance is transferred, the transaction module 150 will notify the biological asset module 110 to provide the traded physical biological cell or tissue to the buyer—the research institution.

According to the above-mentioned embodiments of the NFT transaction method and transaction system, the physical end and the virtual end transactions are conducted in the NFT transaction system 100. The value of the NFT is now determined with the market condition, and the fiat currency price is linked to the market price of the NFT. The NFT therefore reflects the true market value of the biological feature. The problems of NFT being inappropriately hyped or even bubbled can be avoided. In addition, the biological feature from the provider is used to produce unique digital creation, which increases the incentive for the provider to deposit the biological cell or tissue. This mechanism is beneficial to the commercialization of biological features. Moreover, since the transaction is conducted based on blockchain technology and the market condition, the privacy of the provider is protected, the value is properly evaluated, and the transaction process can be safe and transparent.

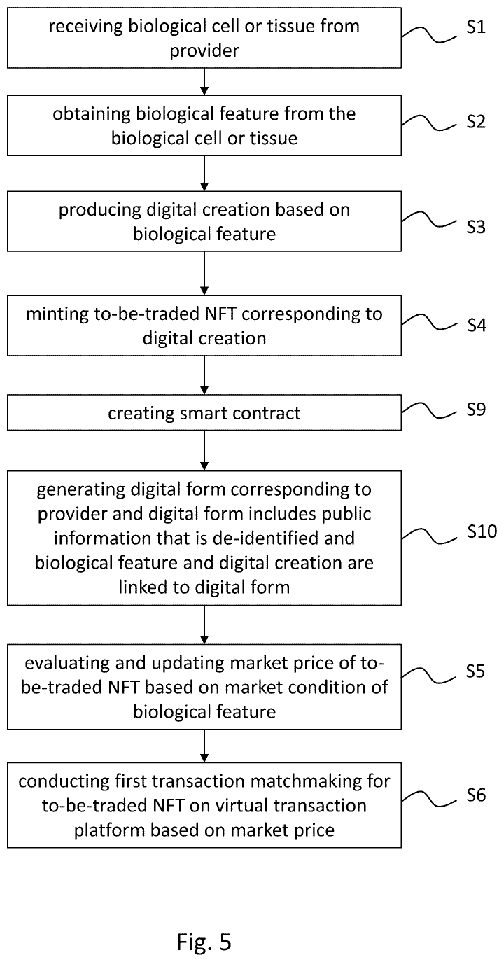

Please refer to FIG. 5, which is a flow chart of an NFT transaction method according to yet another embodiment of the invention. The NFT transaction method is implemented by the NFT transaction system 100 for example. In the present NFT transaction method, steps S1-S4 and steps S5-S6 are the same as those disclosed in the NFT transaction method relating to FIG. 1, and their detail will not be repeated here.

In the present embodiment, the NFT transaction method further includes step S9 and step S10 after step S4.

In step S9, a smart contract is created. When the first transaction matchmaking of step S6 is successful, a profit portion is shared to the provider according to a sharing ratio. One or more regulations for the operations like initializing the to-be-traded NFT, authorizing the to-be-traded NFT, conducting fund transfer, conducting profit sharing, etc. are specified in the smart contract. The smart contract is created to include a monetary flow information that directs to a financial account corresponding to the provider. When the subsequent transaction matchmaking is successful, a portion of the payment to which the seller receives will automatically share to the financial account according to the sharing ratio defined in the smart contract. As a result, the provider can receive a reasonable share of the profit, and the identity of the provider is untraceable during the transaction process, which protects the privacy and ensures the security.

In step S10, a digital form corresponding to the provider is generated. The digital form includes at least one public information that is de-identified, and the biological feature and the digital creation are linked to the digital form. In one embodiment, the digital form at least includes the following information of the provider: medical history, biomedical information, medication description, blood test report, genetic information, test report, or other biomedical and drug-related information. The information in the digital form is disclosed on the virtual transaction platform, so the buyers can search and select appropriate targets. The source of the information in the digital form is the non-identifying information stored in the virtual account of the provider.

The NFT transaction method of the present embodiment can also include the step of conducting a second transaction matchmaking for the biological cell or tissue on the physical transaction platform based on a fiat currency price reflecting the market price. The detail of this step is similar to step S7 relating to FIG. 4 and will not be repeated here. In addition, in the NFT transaction method of the present embodiment, the smart contract is also used for converting the market price into the fiat currency price based on the exchange rate of the virtual transaction platform. The detail of converting the market price is similar to aforementioned NFT transaction method and will not be repeated here as well.

The above-mentioned NFT transaction system 100 is exemplified by including the biological asset module 110, the digital asset module 130, and the transaction module 150. In another embodiment, the NFT transaction system can omit the biological asset module 110.

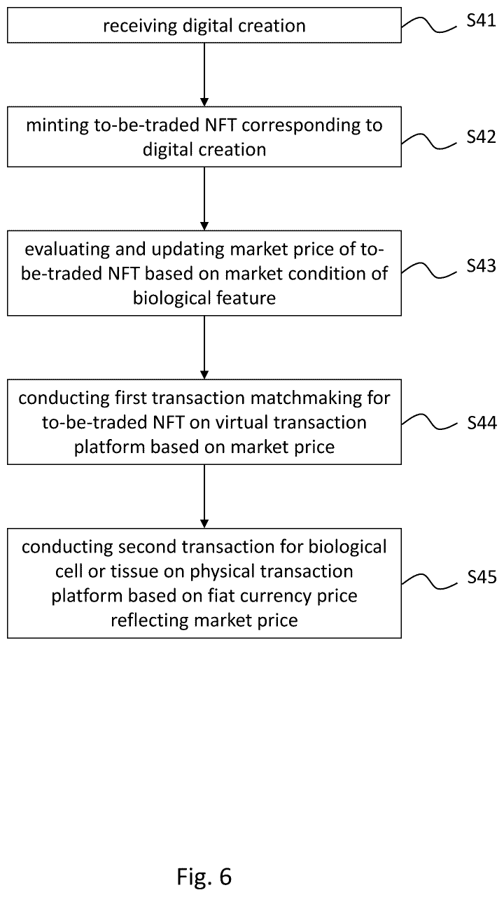

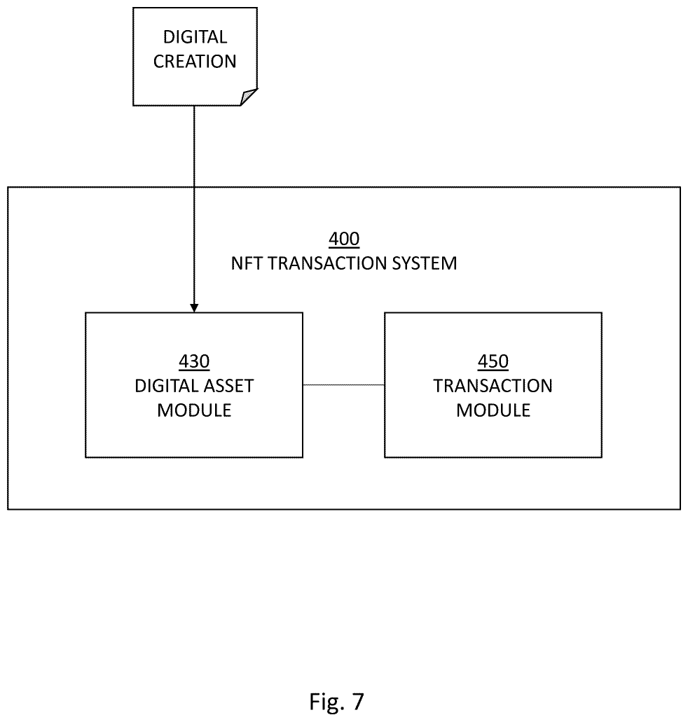

Please refer to FIG. 6 and FIG. 7. FIG. 6 is a flow chart of an NFT transaction method according to a further embodiment of the invention. FIG. 7 is a block diagram of an NFT transaction system according to a further embodiment of the invention.

The NFT transaction system 400 includes a digital asset module 430 and a transaction module 450. The digital asset module 430 is used for receiving a digital creation which is produced based on a biological cell or tissue or a biological feature through a producing method. The digital asset module 430 is further used for minting a to-be-traded NFT corresponding to the digital creation. The transaction module 450 is used for listing the NFT and for evaluating and updating a market price of the NFT based on a market condition of the biological feature. The transaction module 450 is further used for conducting a transaction matchmaking for the NFT on a virtual transaction platform based on the market price. In yet another embodiment, the transaction module 450 is further used for conducting a transaction matchmaking for the biological cell or tissue on a physical transaction platform based on a fiat currency price reflecting the market price.

The NFT transaction method of the present embodiment at least includes the following steps, as shown in FIG. 7.

In step S41, the digital creation which is produced based on the biological feature of the biological cell or tissue is received. Exemplarily, the digital creation is first produced outside the NFT transaction system 400 and then provided to the system 400 to be received by the digital asset module 430.

In step S42, the NFT is minted corresponding to the digital creation.

In step S43, the market price of the NFT is evaluated and updated based on the market condition of the biological feature.

In step S44, the first transaction matchmaking for the NFT is conducted on the virtual transaction platform based on the market price.

Further, the optional step S45 can be performed. In step S45, the second transaction matchmaking for the biological cell or tissue on the physical transaction platform based on the fiat currency price reflecting the market price is conducted.

The details regarding steps S41-S45 are similar to those disclosed in above-mentioned embodiments and will not be repeated here.

The NFT transaction method and the NFT transaction system 400 focus on the minting process of the NFT and the transactions on the virtual transaction platform (in some embodiments further on the physical platform). The source of the digital creation and the producing method thereof are not limited. Any other methods and systems, as long as the information based on the biological cell or tissue or the biological feature is inputted into the digital asset module to mint the NFT, will fall within the scope of the invention.

According to the above elaboration, in the NFT transaction method and transaction system relating to the biological feature of the above-mentioned embodiments of the invention, the NFT is evaluated and traded based on the market condition of the biological feature. The embodiments of the invention at least have the merits such as the NFT's value being match with the market's actual condition, the privacy of the provider can be protected, the transaction process can be transparent and secured, and which are beneficial to the commercialization of the biological feature.

What is claimed is:

1. An NFT transaction method relating to a biological feature, comprising:
(1) receiving, at a biological asset module of an NFT transaction system, a biological cell or tissue from a provider;
(2) obtaining, at the biological asset module, the biological feature from the biological cell or tissue;
(3) producing, at a digital asset module of the NFT transaction system, a digital creation based on the biological feature;
(4) minting, at the digital asset module, a to-be-traded NFT corresponding to the digital creation;
(5) evaluating and updating, at a transaction module of the NFT transaction system, a market price of the to-be-traded NFT based on a market condition of the biological feature; and
(6) conducting a first transaction matchmaking for the to-be-traded NFT on a virtual transaction platform based on the market price.

2. The NFT transaction method according to claim 1, further comprising:
(7) conducting a second transaction matchmaking for the biological cell or tissue on a physical transaction platform based on a fiat currency price reflecting the market price.

3. The NFT transaction method according to claim 2, further comprising:
(8) creating a smart contract for converting the market price into the fiat currency price based on an exchange rate of the virtual transaction platform.

4. The NFT transaction method according to claim 1, further comprising:
(9) creating a smart contract for sharing a profit portion to the provider according to a sharing ratio when the first matchmaking is successful.

5. The NFT transaction method according to claim 4, wherein in step (9), the smart contract is created to comprise a monetary flow information that directs to a financial account corresponding to the provider.

6. The NFT transaction method according to claim 1, further comprising:
(10) generating a digital form corresponding to the provider, wherein the digital form comprises at least one public information that is de-identified, and wherein the biological feature and the digital creation are linked to the digital form.

7. The NFT transaction method according to claim 1, wherein in step (1), the biological cell or tissue comprises blood, cord blood, immune cells, stem cells, skin cells, chondrocytes, cartilage, or cancer tissues.

8. The NFT transaction method according to claim 1, wherein step (3) comprises:
(31) setting a numerical range corresponding to the biological feature;
(32) forming a plurality of creation units, wherein each creation unit comprises one or more numerical values within the numerical range; and
(33) assembling the creation units to form the digital creation.

9. The NFT transaction method according to claim 1, wherein in step (3), the digital creation comprises: a music melody, a digital image, or a digital video.

10. The NFT transaction method according to claim 1, wherein in step (5), the market condition comprises: a search number of the biological feature on the virtual transaction platform, a number of the to-be-traded NFT on the virtual transaction platform, a historical transaction number of the to-be-traded NFT on the virtual transaction platform, or a number of the biological feature corresponding to the provider.

11. The NFT transaction method according to claim 1, wherein in step (4), the biological feature is used as an authentication information for retrieving a blockchain private key of the to-be-traded NFT.

12. The NFT transaction method according to claim 11, further comprising: encrypting the authentication information.

13. An NFT transaction method relating to a biological feature, comprising:
(1) receiving a digital creation which is produced based on the biological feature of a biological cell or tissue;
(2) minting a to-be-traded NFT corresponding to the digital creation;
(3) evaluating and updating a market price of the to-be-traded NFT based on a market condition of the biological feature; and
(4) conducting a first transaction matchmaking for the to-be-traded NFT on a virtual transaction platform based on the market price.

14. The NFT transaction method according to claim 13, further comprising:
(5) conducting a second transaction matchmaking for the biological cell or tissue on a physical transaction platform based on a fiat currency price reflecting the market price.

15. The NFT transaction method according to claim 13, wherein in step (2), the biological feature is used as an authentication information to retrieve a blockchain private key of the to-be-traded NFT.

16. The NFT transaction method according to claim 15, further comprising: encrypting the authentication information.

17. An NFT transaction system relating to a biological feature, comprising:
a digital asset module, for obtaining a digital creation which is produced based on the biological feature of a biological cell or tissue, and for minting a to-be-traded NFT corresponding to the digital creation; and
a transaction module, for listing the to-be-traded NFT, evaluating and updating a market price of the to-be-traded NFT based on a market condition of the biological feature, and conducting a first transaction matchmaking for the to-be-traded NFT based on the market price.

18. The NFT transaction system according to claim 17, wherein the transaction module is further used for conducting a second transaction for the biological cell or tissue based on a fiat currency price reflecting the market price.

19. The NFT transaction system according to claim 17, wherein the digital asset module is further used for configuring the biological feature as an authentication information for retrieving a blockchain private key of the to-be-traded NFT.

20. The NFT transaction system according to claim 19, wherein the digital asset module is further used for encrypting the authentication information.
